(19) United States
(12) Patent Application Publication
(10) Pub. No.: US 2023/0395238 A1
(43) Pub. Date: Dec. 7, 2023

(54) SYSTEM AND METHOD FOR VIRTUAL AND CHEMICAL STAINING OF TISSUE SAMPLES

(71) Applicant: Leica Biosystems Melbourne, Pty Ltd., Mount Waverley (AU)

(72) Inventor: Zbigniew Mioduszewski, Mount Waverley (AU)

(21) Appl. No.: 18/237,192

(22) Filed: Aug. 23, 2023

Related U.S. Application Data
(63) Continuation of application No. PCT/US2022/015517, filed on Feb. 7, 2022.
(60) Provisional application No. 63/154,548, filed on Feb. 26, 2021.

(51) Int. Cl. G16H 30/40; G16H 50/20; G06T 7/00; G01N 1/30

(52) U.S. Cl. CPC G16H 30/40; G16H 50/20; G06T 7/0012; G01N 1/30; G06T 2207/20081; G06T 2207/30024

(57) ABSTRACT

Systems and methods for hybrid virtual and chemical staining of tissue samples are disclosed. In one aspect, an image analysis apparatus includes a memory coupled to an imaging device, and a hardware processor coupled to the memory. The hardware processor is configured to receive image data from the imaging device, the image data representative of a tissue sample in a first state, and perform virtual staining of the tissue sample based on the image data to generate one or more virtual stained images of the tissue sample. The hardware processor is further configured to order chemical staining of the tissue sample in the first state, receive one or more chemically stained images, and generate a set of the one or more virtual stained images of the tissue sample from the virtual staining and the one or more chemically stained images of the tissue sample from the chemical staining.

### CROSS-REFERENCE TO RELATED APPLICATION(S)

This application claims the benefit of U.S. Provisional Patent Application No. 63/154,548, filed Feb. 26, 2021, the disclosure of which is incorporated herein by reference.

### BACKGROUND

### Technical Field

The described technology relates to histology, and in particular, techniques for hybrid virtual and chemical staining of tissue samples.

### Description of the Related Technology

Tissue samples can be analyzed under a microscope for various diagnostic purposes, including detecting cancer by identifying structural abnormalities in the tissue sample. A tissue sample can be imaged to produce image data using a microscope or other optical system. Developments within the field of tissue sample diagnostics include the use of optical imaging techniques to “virtually” stain a tissue sample without using chemical stains. Such developments may enable improvements to the histology workflow, which may result in shortening the overall time between obtaining the tissue sample and arriving at a diagnosis.

### SUMMARY

In one aspect, there is provided image analysis apparatus, comprising: a memory coupled to an imaging device; and a hardware processor coupled to the memory and configured to: receive image data from the imaging device, the image data representative of a tissue sample in a first state, perform virtual staining of the tissue sample based on the image data to generate one or more virtual stained images of the tissue sample, order chemical staining of the tissue sample in the first state, receive one or more chemically stained images, and generate a set of the one or more virtual stained images of the tissue sample from the virtual staining and the one or more chemically stained images of the tissue sample from the chemical staining.

The hardware processor can be further configured to: generate a first diagnosis based on the virtual staining of the tissue sample in the first state, the first diagnosis comprising the one or more virtual stained images of the tissue sample.

The hardware processor can be further configured to: determine at least one assay for the chemical staining of the tissue sample in the first state, wherein the order of the chemical staining includes an indication of the at least one assay to be used in the chemical staining of the tissue sample.

The hardware processor can be further configured to: execute a machine learning algorithm using the virtual stained images of the tissue sample as an input, the machine learning algorithm configured to generate a first diagnosis comprising an indication of the disease based on the tissue sample.

The hardware processor can be further configured to: generate a first diagnosis based on the virtual staining of the tissue sample in the first state, wherein the chemical staining of the tissue sample using the at least one assay is configured to differentiate between different types of the disease indicated by the first diagnosis.

The hardware processor can be further configured to: execute a machine learning algorithm using the virtual stained images of the tissue sample as an input, the machine learning algorithm configured to generate a first diagnosis comprising an indication of the disease based on the tissue sample, wherein the machine learning algorithm is configured to follow a decision tree that selects the at least one assay based on the disease indicated by the first diagnosis.

The identifying and/or ordering of the chemical staining of the tissue sample can be performed automatically in response to a machine learning or artificial intelligence algorithm generating a first diagnosis.

The chemical staining can be performed on the same tissue sample used in the virtual staining.

The hardware processor can be further configured to perform the virtual staining and the generating of the set of the one or more images without storing the tissue sample.

The imaging device can be configured to generate the image data using coverslipless imaging, and the chemical staining can be imaged using coverslipless imaging.

In another aspect, there is provided a method of diagnosing a disease based on a tissue sample, comprising: performing virtual staining of the tissue sample in a first state to generate one or more virtual stained images of the tissue sample; generating a first diagnosis based on the virtual staining of the tissue sample in the first state, the first diagnosis comprising the one or more virtual stained images of the tissue sample; determining, based on the first diagnosis, at least one assay for chemical staining of the tissue sample in the first state; and generating a set of the one or more virtual stained images of the tissue sample from the virtual staining and one or more chemical stained images of the tissue sample from the chemical staining.

The performing the virtual staining of the tissue sample can comprise: providing the virtual stained images of the tissue sample to a machine learning algorithm, wherein the machine learning algorithm is configured to generate the first diagnosis comprising an indication of the disease based on the tissue sample.

The chemical staining can be performed on the same tissue sample used in the virtual staining.

The virtual staining and the generating the set of the one or more images can be performed without storing the tissue sample.

The method can further comprise: generating image data of the tissue sample using an image device, wherein the image data generated by the image device is used as an input for the virtual staining.

The generating of the image data and the chemical staining can be performed using coverslipless imaging.

In yet another aspect, there is provided an image analysis apparatus, comprising: a memory coupled to an imaging device; and a hardware processor coupled to the memory and configured to: obtain image data from the imaging device, the image data representative of a tissue sample, perform virtual staining of the tissue sample based on the image data to generate one or more virtual stained images of the tissue sample, and obtain one or more images of the same tissue sample having a chemical stain.

The tissue sample can be directed to undergo the chemical stain after the hardware processor performs virtual staining of the tissue sample.

The hardware processor can be further configured to cause the ordering of the chemical staining of the tissue based on the one or more virtual stained images of the tissue sample.

The hardware processor can be further configured to generate a first diagnosis based on the virtual staining of the tissue sample.

In still yet another aspect, there is provided a method of processing a tissue sample, the method comprising: obtaining an image of a tissue sample with a chemical stain, after obtaining an image of the same tissue sample with a virtual stain and without a chemical stain.

### BRIEF DESCRIPTION OF THE DRAWINGS

The features and advantages of the multi-stage stop devices, systems, and methods described herein will become more fully apparent from the following description and appended claims, taken in conjunction with the accompanying drawings. These drawings depict several embodiments in accordance with the disclosure and are not to be considered limiting of its scope. In the drawings, similar reference numbers or symbols typically identify similar components, unless context dictates otherwise. The drawings may not be drawn to scale.

FIG. 1 illustrates an example environment in which a user and/or an imaging system may implement an image analysis system according to some embodiments.

FIG. 2 depicts an example workflow for generating image data from a tissue sample block according to some embodiments.

FIG. 3A illustrates an example prepared tissue block according to some embodiments.

FIG. 3B illustrates an example prepared tissue block and an example prepared tissue slice according to some embodiments.

FIG. 4 shows an example imaging device, according to one embodiment.

FIG. 5 is an example computing system which can implement any one or more imaging devices, image analysis system, and user computing device of the multispectral imaging system illustrated in FIG. 1.

FIG. 6 depicts a schematic diagram of a machine learning algorithm, including a multiple layer neural network in accordance with aspects of the present disclosure.

FIG. 7 is an example method for hybrid virtual and chemical staining of tissue samples in accordance with aspects of this disclosure.

FIG. 8 is an example method for diagnosing and typing a disease in accordance with aspects of this disclosure.

### DETAILED DESCRIPTION

The features of the systems and methods for hybrid virtual and chemical staining of tissue samples will now be described in detail with reference to certain embodiments illustrated in the figures. The illustrated embodiments described herein are provided by way of illustration and are not meant to be limiting. Other embodiments can be utilized, and other changes can be made, without departing from the spirit or scope of the subject matter presented. It will be readily understood that the aspects and features of the present disclosure described below and illustrated in the figures can be arranged, substituted, combined, and designed in a wide variety of different configurations by a person of ordinary skill in the art, all of which are made part of this disclosure.

The diagnosis of tissue samples may involve several processing steps to prepare the tissue sample for viewing under a microscope. While traditional diagnostics techniques may involve staining a tissue sample to provide additional visual contrast to the cellular structure of the sample when viewed under a microscope and manually diagnosing a disease by viewing the stained image through the microscope, optical scanning on the sample can be used to create image data which can be “virtually” stained using an image analysis system and provided to an image analysis system for processing. In certain implementations, the optical scanning may be performed using multispectral imaging (also referred to as multispectral optical scanning) to provide additional information compared to optical scanning using a single frequency of light. In some implementations, the image analysis system can include a machine learning or artificial intelligence algorithm trained to identify and diagnose one or more diseases by identifying structures or features present in the image data that are consistent with training data used to train the machine learning algorithm.

Multispectral imaging may involve providing multispectral light to the tissue sample using a multispectral light source and detecting light emitted from the sample in response to the multispectral light using an imaging sensor. Under certain wavelengths/frequencies of the multispectral light, the tissue sample may exhibit autofluorescence which can be detected to generate image data that can be virtually stained. The use of virtual staining of tissue samples may enable various improvements in the histology workflow. For example, image data produced during virtual staining can be provided to a machine learning algorithm (also referred to as an artificial intelligence “AI” algorithm) which can be trained to provide a diagnosis of a disease present in the tissue sample.

However, there may be limitations to the data that can be obtained using only virtual staining. That is, while virtual staining may be able to produce markers that are substantially similar to certain chemical stains (e.g., hematoxylin and eosin (H&E) stains), markers which are produced using other chemical stains (e.g., immunohistochemistry (IHC) stains) may not be easily achieved using virtual staining. Thus, it may still be necessary to apply chemical stains to a tissue sample in order to fully diagnose a disease.

As used herein, chemical staining generally refers to the physical staining of a tissue sample using an assay in order to provide additional visual contrast to certain aspects of the cellular structure of the tissue sample. There are at least three there common types of chemical stains that are used in addition to H&E staining. Any one or more of the below example types of chemical stains, or other types of chemical stains not explicitly listed below, may be used in accordance with aspects of this disclosure.

The first type of chemical stain is termed a “special stain,” which typically involves washing one or more chemical dyes the tissue sample in order to highlight certain features of interest (e.g., bacteria and/or fungi) or to enable contrast for viewing of cell morphology and/or tissue structures (e.g., highlighting carbohydrate deposits).

The second type of chemical stain is termed immunohistochemistry (IHC), and typically involves using antibody markers to identify particular proteins within the tissue sample. These antibodies can be highlighted using visible, fluorescent, and/or other detection methods.

The third type of chemical stain may be termed molecular testing (e.g., in situ hybridization (ISH)), and typically involves using an assay to identify specific DNA or RNA mutations in the genome. These mutations can also be highlighted using visible, fluorescent, and/or other detection methods.

With traditional histology workflow, the total length of time between a tissue biopsy and the time at which a pathologist is able to determine the final diagnosis of a disease present in the tissue sample is typically greater than the length of time between a virtual staining and a final diagnosis. For example, traditional histology may involve first obtaining the tissue sample (e.g., via a biopsy) and performing an initial stain on at least one slice of the tissue sample (e.g., an H&E stain) at a lab. After the initial stain, the remainder of the tissue sample from which the slice was obtained is typically stored to preserve the tissue sample for further staining. Storing the tissue sample and retrieving the stored tissue sample for chemical staining may involve additional steps performed at the lab, increasing the length of time between the tissue biopsy and the final diagnosis.

The lab can produce one or more images based on the stained tissue sample which are typically sent to the pathologist at the end of the day. The pathologist reviews the image of the stained slide, and based on an initial diagnosis of the slide, may order one or more other chemical stains to aid in the diagnosis. The lab receives the orders, retrieves the stored tissue sample, and performs the ordered chemical stains on new slices of the tissue sample, and sends the subsequent stained slides to the pathologist. In other implementations, digital images of the stained slides may be sent to the pathologist in addition to or in place of the physical slides. After receiving the slides/images, the pathologist can complete the diagnosis using the images produced based on both sets of stained slides. However, it can be difficult for the pathologist to mentally matching similar features on different sections/slides because the features may be aligned differently due to the necessity of staining separate slices of the tissue sample.

Although the total length of active time involved in the histological workflow may be less than about 24 hours, due to the downtime associated with transmitting images between the lab and the pathologist, along with scheduling the time of the lab technician and the pathologist, the amount of real time elapsed between taking the biopsy and final diagnosis range from about one week for simple cases to about 50 days on average or longer for more complex diagnoses. It is desirable to reduce the time between taking the biopsy and the final diagnosis without significantly altering the scheduling demands on the lab technician or the pathologist.

Aspects of this disclosure relate to systems and methods for hybrid virtual and chemical staining of tissue samples which can address one or more of the issues relating to timing and workflow. Advantageously, aspects of this disclosure can use both virtual and chemical staining in the histology workflow, which may significantly reduce the amount of time required to arrive at the final diagnosis.

### System Overview

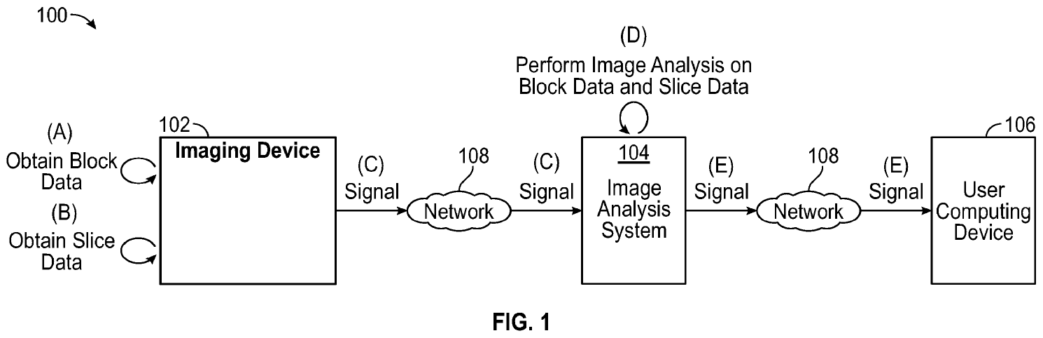

FIG. 1 illustrates an example environment 100 (e.g., a hybrid virtual and chemical staining system) in which a user and/or the multispectral imaging system may implement an image analysis system 104 according to some embodiments. The image analysis system 104 may perform image analysis on received image data. The image analysis system 104 can perform virtual staining on the image data obtained using multispectral imaging for input to a machine learning algorithm. Based on image data generated during virtual staining, the machine learning algorithm can generate a first diagnosis which may include an indication of whether the image data is indicative of a disease present in the tissue sample.

The image analysis system 104 may perform the image analysis using an image analysis module (not shown in FIG. 1). The image analysis system 104 may receive the image data from an imaging device 102 and transmit the recommendation to a user computing device 106 for processing. Although some examples herein refer to a specific type of device as being the imaging device 102, the image analysis system 104, or the user computing device 106, the examples are illustrative only and are not intended to be limiting, required, or exhaustive. The image analysis system 104 may be any type of computing device (e.g., a server, a node, a router, a network host, etc.). Further, the imaging device 102 may be any type of imaging device (e.g., a camera, a scanner, a mobile device, a laptop, etc.). In some embodiments, the imaging device 102 may include a plurality of imaging devices. Further, the user computing device 106 may be any type of computing device (e.g., a mobile device, a laptop, etc.).

In some implementations, the imaging device 102 includes a light source 102a configured to emit multispectral light onto the tissue sample(s) and the image sensor 102b configured to detect multispectral light emitted from the tissue sample. The multispectral imaging using the light source 102a can involve providing light to the tissue sample carried by a carrier within a range of frequencies. That is, the light source 102a may be configured to generate light across a spectrum of frequencies to provide multispectral imaging.

In certain embodiments, the tissue sample may reflect light received from the light source 102a, which can then be detected at the image sensor 102b. In these implementations, the light source 102a and the image sensor 102b may be located on substantially the same side of the tissue sample. In other implementations, the light source 102a and the image sensor 102b may be located on opposing sides of the tissue sample. The image sensor 102b may be further configured to generate image data based on the multispectral light detected at the image sensor 102b. In certain implementations, the image sensor 102b may include a high-resolution sensor configured to generate a high-resolution image of the tissue sample. The high-resolution image may be generated based on excitation of the tissue sample in response to laser light emitted onto the sample at different frequencies (e.g., a frequency spectrum).

The imaging device 102 may capture and/or generate image data for analysis. The imaging device 102 may include one or more of a lenses, an image sensor, a processor, or memory. The imaging device 102 may receive a user interaction. The user interaction may be a request to capture image data. Based on the user interaction, the imaging device 102 may capture image data. In some embodiments, the imaging device 102 may capture image data periodically (e.g., every 10, 20, or 30 minutes). In other embodiments, the imaging device 102 may determine that an item has been placed in view of the imaging device 102 (e.g., a histological sample has been placed on a table and/or platform associated with the imaging device 102) and, based on this determination, capture image data corresponding to the item. The imaging device 102 may further receive image data from additional imaging devices. For example, the imaging device 102 may be a node that routes image data from other imaging devices to the image analysis system 104. In some embodiments, the imaging device 102 may be located within the image analysis system 104. For example, the imaging device 102 may be a component of the image analysis system 104. Further, the image analysis system 104 may perform an imaging function. In other embodiments, the imaging device 102 and the image analysis system 104 may be connected (e.g., wirelessly or wired connection). For example, the imaging device 102 and the image analysis system 104 may communicate over a network 108. Further, the imaging device 102 and the image analysis system 104 may communicate over a wired connection. In one embodiment, the image analysis system 104 may include a docking station that enables the imaging device 102 to dock with the image analysis system 104. An electrical contact of the image analysis system 104 may connect with an electrical contact of the imaging device 102. The image analysis system 104 may be configured to determine when the imaging device 102 has been connected with the image analysis system 104 based at least in part on the electrical contacts of the image analysis system 104. In some embodiments, the image analysis system 104 may use one or more other sensors (e.g., a proximity sensor) to determine that an imaging device 102 has been connected to the image analysis system 104. In some embodiments, the image analysis system 104 may be connected to (via a wired or a wireless connection) a plurality of imaging devices.

The image analysis system 104 may include various components for providing the features described herein. In some embodiments, the image analysis system 104 may include one or more image analysis modules to perform the image analysis of the image data received from the imaging device 102. The image analysis modules may perform one or more imaging algorithms using the image data.

The image analysis system 104 may be connected to the user computing device 106. The image analysis system 104 may be connected (via a wireless or wired connection) to the user computing device 106 to provide a recommendation for a set of image data. The image analysis system 104 may transmit the recommendation to the user computing device 106 via the network 108. In some embodiments, the image analysis system 104 and the user computing device 106 may be configured for connection such that the user computing device 106 can engage and disengage with image analysis system 104 in order to receive the recommendation. For example, the user computing device 106 may engage with the image analysis system 104 upon determining that the image analysis system 104 has generated a recommendation for the user computing device 106. Further, a particular user computing device 106 may connect to the image analysis system 104 based on the image analysis system 104 performing image analysis on image data that corresponds to the particular user computing device 106. For example, a user may be associated with a plurality of histological samples. Upon determining, that a particular histological sample is associated with a particular user and a corresponding user computing device 106, the image analysis system 104 can transmit a recommendation for the histological sample to the particular user computing device 106. In some embodiments, the user computing device 106 may dock with the image analysis system 104 in order to receive the recommendation.

In some implementations, the imaging device 102, the image analysis system 104, and/or the user computing device 106 may be in wireless communication. For example, the imaging device 102, the image analysis system 104, and/or the user computing device 106 may communicate over a network 108. The network 108 may include any viable communication technology, such as wired and/or wireless modalities and/or technologies. The network may include any combination of Personal Area Networks (“PANs”), Local Area Networks (“LANs”), Campus Area Networks (“CANs”), Metropolitan Area Networks (“MANs”), extranets, intranets, the Internet, short-range wireless communication networks (e.g., ZigBee, Bluetooth, etc.), Wide Area Networks (“WANs”)—both centralized and/or distributed—and/or any combination, permutation, and/or aggregation thereof. The network 108 may include, and/or may or may not have access to and/or from, the internet. The imaging device 102 and the image analysis system 104 may communicate image data. For example, the imaging device 102 may communicate image data associated with a histological sample to the image analysis system 104 via the network 108 for analysis. The image analysis system 104 and the user computing device 106 may communicate a recommendation corresponding to the image data. For example, the image analysis system 104 may communicate a diagnosis regarding whether the image data is indicative of a disease present in the tissue sample based on the results of a machine learning algorithm. In some embodiments, the imaging device 102 and the image analysis system 104 may communicate via a first network and the image analysis system 104 and the user computing device 106 may communicate via a second network. In other embodiments, the imaging device 102, the image analysis system 104, and the user computing device 106 may communicate over the same network.

With reference to an illustrative embodiment, at [A], the imaging device 102 can obtain block data. In order to obtain the block data, the imaging device 102 can image (e.g., scan, capture, record, etc.) a tissue block. The tissue block may be a histological sample. For example, the tissue block may be a block of biological tissue that has been removed and prepared for analysis. As will be discussed in further below, in order to prepare the tissue block for analysis, various histological techniques may be performed on the tissue block. The imaging device 102 can capture an image of the tissue block and store corresponding block data in the imaging device 102. The imaging device 102 may obtain the block data based on a user interaction. For example, a user may provide an input through a user interface (e.g., a graphical user interface (“GUI”)) and request that the imaging device 102 image the tissue block. Further, the user can interact with imaging device 102 to cause the imaging device 102 to image the tissue block. For example, the user can toggle a switch of the imaging device 102, push a button of the imaging device 102, provide a voice command to the imaging device 102, or otherwise interact with the imaging device 102 to cause the imaging device 102 to image the tissue block. In some embodiments, the imaging device 102 may image the tissue block based on detecting, by the imaging device 102, that a tissue block has been placed in a viewport of the imaging device 102. For example, the imaging device 102 may determine that a tissue block has been placed on a viewport of the imaging device 102 and, based on this determination, image the tissue block.

At [B], the imaging device 102 can obtain slice data. In some embodiments, the imaging device 102 can obtain the slice data and the block data. In other embodiments, a first imaging device can obtain the slice and a second imaging device can obtain the block data. In order to obtain the slice data, the imaging device 102 can image (e.g., scan, capture, record, etc.) a slice of the tissue block. The slice of the tissue block may be a slice of the histological sample. For example, the tissue block may be sliced (e.g., sectioned) in order to generate one or more slices of the tissue block. In some embodiments, a portion of the tissue block may be sliced to generate a slice of the tissue block such that a first portion of the tissue block corresponds to the tissue block imaged to obtain the block data and a second portion of the tissue block corresponds to the slice of the tissue block imaged to obtain the slice data. As will be discussed in further detail below, various histological techniques may be performed on the tissue block in order to generate the slice of the tissue block. The imaging device 102 can capture an image of the slice and store corresponding slice data in the imaging device 102. The imaging device 102 may obtain the slice data based on a user interaction. For example, a user may provide an input through a user interface and request that the imaging device 102 image the slice. Further, the user can interact with imaging device 102 to cause the imaging device 102 to image the slice. In some embodiments, the imaging device 102 may image the tissue block based on detecting, by the imaging device 102, that the tissue block has been sliced or that a slice has been placed in a viewport of the imaging device 102.

At [C], the imaging device 102 can transmit a signal to the image analysis system 104 representing the captured image data (e.g., the block data and the slice data). The imaging device 102 can send the captured image data as an electronic signal to the image analysis system 104 via the network 108. The signal may include and/or correspond to a pixel representation of the block data and/or the slice data. It will be understood that the signal can include and/or correspond to more, less, or different image data. For example, the signal may correspond to multiple slices of a tissue block and may represent a first slice data and a second slice data. Further, the signal may enable the image analysis system 104 to reconstruct the block data and/or the slice data. In some embodiments, the imaging device 102 can transmit a first signal corresponding to the block data and a second signal corresponding to the slice data. In other embodiments, a first imaging device can transmit a signal corresponding to the block data and a second imaging device can transmit a signal corresponding to the slice data.

At [D], the image analysis system 104 can perform image analysis on the block data and the slice data provided by the imaging device 102. In order to perform the image analysis, the image analysis system 104 may utilize one or more image analysis modules that can perform one or more image processing functions. For example, the image analysis module may include an imaging algorithm, a machine learning model, a convolutional neural network, or any other modules for performing the image processing functions. Based on performing the image processing functions, the image analysis module can determine a likelihood that the block data and the slice data correspond to the same tissue block. For example, an image processing functions may include an edge analysis of the block data and the slice data and based on the edge analysis, determine whether the block data and the slice data correspond to the same tissue block. The image analysis system 104 can obtain a confidence threshold from the user computing device 106, the imaging device 102, or any other device. In some embodiments, the image analysis system 104 can determine the confidence threshold based on a response by the user computing device 106 to a particular recommendation. Further, the confidence threshold may be specific to a user, a group of users, a type of tissue block, a location of the tissue block, or any other factor. The image analysis system 104 can compare the determined confidence threshold with the image analysis performed by the image analysis module. For example, the image analysis system 104 can provide a diagnosis regarding whether the image data is indicative of a disease present in the tissue sample, for example, based on the results of a machine learning algorithm.

At [E], the image analysis system 104 can transmit a signal to the user computing device 106. The image analysis system 104 can send the signal as an electrical signal to the user computing device 106 via the network 108. The signal may include and/or correspond to a representation of the diagnosis. Based on receiving the signal, the user computing device 106 can determine the diagnosis. In some embodiments, the image analysis system 104 may transmit a series of recommendations corresponding to a group of tissues blocks and/or a group of slices. The image analysis system 104 can include, in the recommendation, a recommended action of a user. For example, the recommendation may include a recommendation for the user to review the tissue block and the slice. Further, the recommendation may include a recommendation that the user does not need to review the tissue block and the slice.

### Imaging Prepared Blocks and Prepared Slices

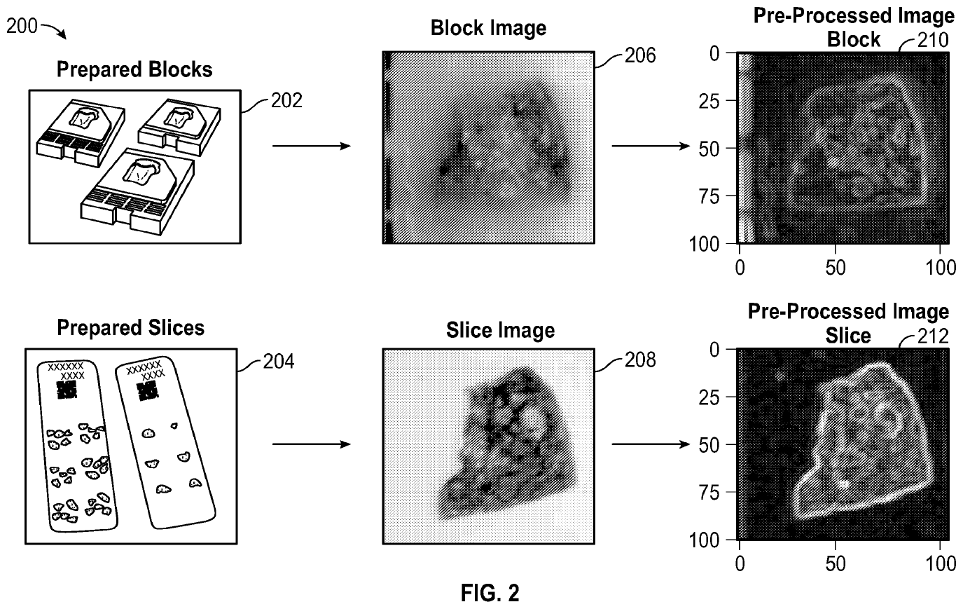

FIG. 2 depicts an example workflow 200 for generating image data from a tissue sample block according to some embodiments. The example workflow 200 illustrates a process for generating prepared blocks and prepared slices from a tissue block and generating pre-processed images based on the prepared blocks and the prepared slices. The example workflow 200 may be implemented by one or more computing devices. For example, the example workflow 200 may be implemented by a microtome, a coverslipper, a stainer, and an imaging device. Each computing device may perform a portion of the example workflow. For example, the microtome may cut the tissue block in order to generate one or more slices of the tissue block. The coverslipper or microtome may be used to create a first slide for the tissue block and/or a second slide for a slice of the tissue block, the stainer may stain each slide, and the imaging device may image each slide.

A tissue block can be obtained from a patient (e.g., a human, an animal, etc.). The tissue block may correspond to a section of tissue from the patient. The tissue block may be surgically removed from the patient for further analysis. For example, the tissue block may be removed in order to determine if the tissue block has certain characteristics (e.g., if the tissue block is cancerous). In order to generate the prepared blocks 202, the tissue block may be prepared using a particular preparation process by a tissue preparer. For example, the tissue block may be preserved and subsequently embedded in a paraffin wax block. Further, the tissue block may be embedded (in a frozen state or a fresh state) in a block. The tissue block may also be embedded using an optimal cutting temperature (“OCT”) compound. The preparation process may include one or more of a paraffin embedding, an OCT-embedding, or any other embedding of the tissue block. In the example of FIG. 2, the tissue block is embedded using paraffin embedding. Further, the tissue block is embedded within a paraffin wax block and mounted on a microscopic slide in order to formulate the prepared block.

The microtome can obtain a slice of the tissue block in order to generate the prepared slices 204. The microtome can use one or more blades to slice the tissue block and generate a slice (e.g., a section) of the tissue block. The microtome can further slice the tissue block to generate a slice with a preferred level of thickness. For example, the slice of the tissue block may be 1 millimeter. The microtome can provide the slice of the tissue block to a coverslipper. The coverslipper can encase the slice of the tissue block in a slide to generate the prepared slices 204. The prepared slices 204 may include the slice mounted in a certain position. Further, in generating the prepared slices 204, a stainer may also stain the slice of the tissue block using any staining protocol. Further, the stainer may stain the slice of the tissue block in order to highlight certain portions of the prepared slices 204 (e.g., an area of interest). In some embodiments, a computing device may include both the coverslipper and the stainer and the slide may be stained as part of the process of generating the slide.

The prepared blocks 202 and the prepared slices 204 may be provided to an imaging device for imaging. In some embodiments, the prepared blocks 202 and the prepared slices 204 may be provided to the same imaging device. In other embodiments, the prepared blocks 202 and the prepared slices 204 are provided to different imaging devices. The imaging device can perform one or more imaging operations on the prepared blocks 202 and the prepared slices 204. In some embodiments, a computing device may include one or more of the tissue preparer, the microtome, the coverslipper, the stainer, and/or the imaging device.

The imaging device can capture an image of the prepared block 202 in order to generate the block image 206. The block image 206 may be a representation of the prepared block 202. For example, the block image 206 may be a representation of the prepared block 202 from one direction (e.g., from above). The representation of the prepared block 202 may correspond to the same direction as the prepared slices 204 and/or the slice of the tissue block. For example, if the tissue block is sliced in a cross-sectional manner in order to generate the slice of the tissue block, the block image 206 may correspond to the same cross-sectional view. In order to generate the block image 206, the prepared block 202 may be placed in a cradle of the imaging device and imaged by the imaging device. Further, the block image 206 may include certain characteristics. For example, the block image 206 may be a color image with a particular resolution level, clarity level, zoom level, or any other image characteristics.

The imaging device can capture an image of the prepared slices 204 in order to generate the slice image 208. The imaging device can capture an image of a particular slice of the prepared slices 204. For example, a slide may include any number of prepared slices and the imaging device may capture an image of a particular slice of the prepared slices. The slice image 208 may be a representation of the prepared slices 204. The slice image 208 may correspond to a view of the slice according to how the slice of the tissue block was generated. For example, if the slice of the tissue block was generated via a cross-sectional cut of the tissue block, the slice image 208 may correspond to the same cross-sectional view. In order to generate the slice image 208, the slide containing the prepared slices 204 may be placed in a cradle of the imaging device (e.g., in a viewer of a microscope) and imaged by the imaging device. Further, the slice image 208 may include certain characteristics. For example, the slice image 208 may be a color image with a particular resolution level, clarity level, zoom level, or any other image characteristics.

The imaging device can process the block image 206 in order to generate a pre-processed image 210 and the slice image 208 in order to generate the pre-processed image 212. The imaging device can perform one or more image operations on the block image 206 and the slice image 208 in order to generate the pre-processed image 210 and the pre-processed image 212. The one or more image operations may include isolating (e.g., focusing on) various features of the pre-processed image 210 and the pre-processed imaged 212. For example, the one or more image operations may include isolating the edges of a slice or a tissue block, isolating areas of interest within a slice or a tissue block, or otherwise modifying (e.g., transforming) the block image 206 and/or the slice image 208. In some embodiments, the imaging device can perform the one or more image operations on one of the block image 206 or the slice image 208. For example, the imaging may perform the one or more image operations on the block image 206. In other embodiments, the imaging device can perform first image operations on the block image 206 and second image operations on the slice image 208. The imaging device may provide the pre-processed image 210 and the pre-processed image 212 to the image analysis system to determine a likelihood that the pre-processed image 210 and the pre-processed image 212 correspond to the same tissue block.

### Slicing a Tissue Block

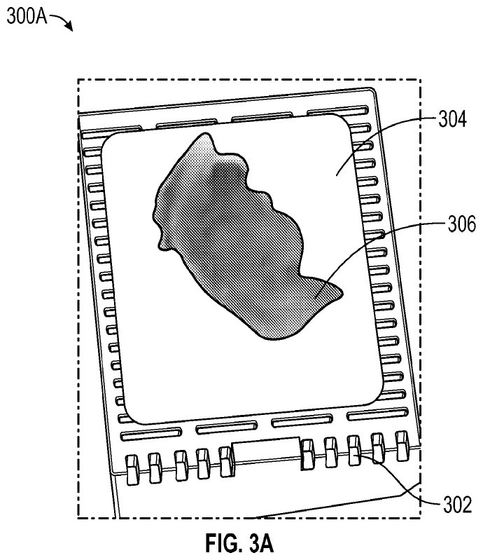

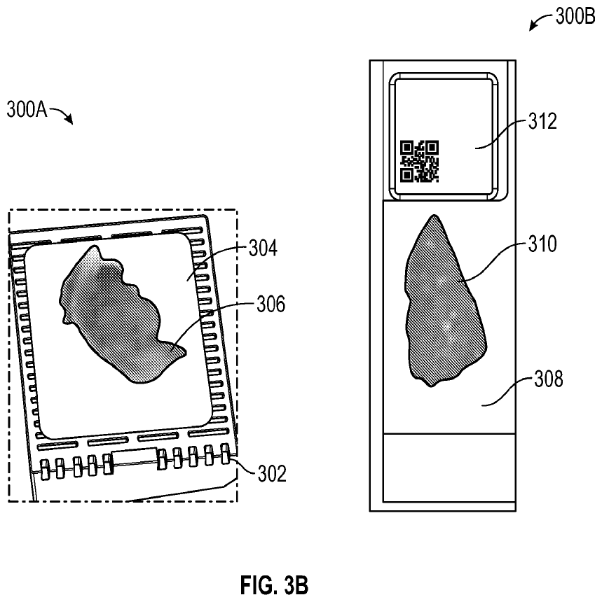

FIG. 3A illustrates an example prepared tissue block 300A according to some embodiments. The prepared tissue block 300A may include a tissue block 306 that is preserved (e.g., chemically preserved, fixed, supported) in a particular manner. In order to generate the prepared tissue block 300A, the tissue block 306 can be placed in a fixing agent (e.g., a liquid fixing agent). For example, the tissue block 306 can be placed in a fixative such as formaldehyde solution. The fixing agent can penetrate the tissue block 306 and preserve the tissue block 306. The tissue block 306 can subsequently be isolated in order to enable further preservation of the tissue block 306. Further, the tissue block 306 can be immersed in one or more solutions (e.g., ethanol solutions) in order to replace water within the tissue block 306 with the one or more solutions. The tissue block 306 can be immersed in one or more intermediate solutions. Further, the tissue block 306 can be immersed in a final solution (e.g., a histological wax). For example, the histological wax may be a purified paraffin wax. After being immersed in a final solution, the tissue block 306 may be formed into a prepared tissue block 300A. For example, the tissue block 306 may be placed into a mould filled with the histological wax. By placing the tissue block in the mould, the tissue block 306 may be moulded (e.g., encased) in the final solution 304. In order to generate the prepared tissue block 300A, the tissue block 306 in the final solution 304 may be placed on a platform 302. Therefore, the prepared tissue block 300A may be generated. It will be understood that the prepared tissue block 300A may be prepared according to any tissue preparation methods.

FIG. 3B illustrates an example prepared tissue block 300A and an example prepared tissue slice 300B according to some embodiments. The prepared tissue block 300A may include the tissue block 306 encased in a final solution 304 and placed on a platform 302. In order to generate the prepared tissue slice 300B, the prepared tissue block 300A may be sliced by a microtome. The microtome may include one or more blades to slice the prepared tissue block 300A. The microtome may take a cross-sectional slice 310 of the prepared tissue block 300A using the one or more blades. The cross-sectional slice 310 of the prepared tissue block 300A may include a slice 310 (e.g., a section) of the tissue block 306 encased in a slice of the final solution 304. In order to preserve the slice 310 of the tissue block 306, the slice 310 of the tissue block 306 may be modified (e.g., washed) to remove the final solution 304 from the slice 310 of the tissue block 306. For example, the final solution 304 may be rinsed and/or isolated from the slice 310 of the tissue block 306. Further, the slice 310 of the tissue block 306 may be stained by a stainer. In some embodiments, the slice 310 of the tissue block 306 may not be stained. The slice 310 of the tissue block 306 may subsequently be encased in a slide 308 by a coverslipper to generate the prepared tissue slice 300B. The prepared tissue slice 300B may include an identifier 312 identifying the tissue block 306 that corresponds to the prepared tissue slice 300B. Not shown in FIG. 3B, the prepared tissue block 300A may also include an identifier that identifies the tissue block 306 that corresponds to the prepared tissue block 300A. As the prepared tissue block 300A and the prepared tissue slice 300B correspond to the same tissue block 306, the identifier of the prepared tissue block 300A and the identifier 312 of the prepared tissue slice 300B may identify the same tissue block 306.

### Imaging Devices

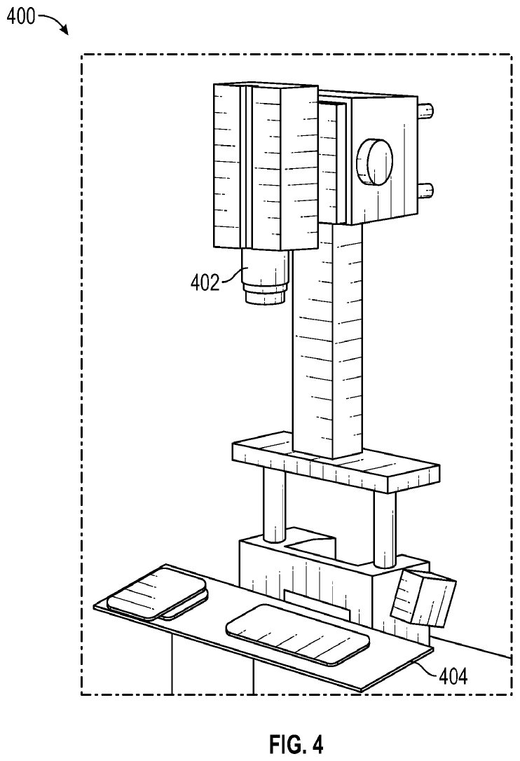

FIG. 4 shows an example imaging device 400, according to one embodiment. The imaging device 400 can include an imaging apparatus 402 (e.g., a lens and an image sensor) and a platform 404. The imaging device 400 can receive a prepared tissue block and/or a prepared tissue slice via the platform 404. Further, the imaging device can use the imaging apparatus 402 to capture image data corresponding to the prepared block and/or the prepared slice. The imaging device 400 can be one or more of a camera, a scanner, a medical imaging device, etc. Further, the imaging device 400 can use imaging technologies such as X-ray radiography, magnetic resonance imaging, ultrasound, endoscopy, elastography, tactile imaging, thermography, medical photography, nuclear medicine functional imaging, positron emission tomography, single-photon emission computed tomography, etc. For example, the imaging device can be a magnetic resonance imaging (“MRI”) scanner, a positron emission tomography (“PET”) scanner, an ultrasound imaging device, an x-ray imaging device, a computerized tomography (“CT”) scanner,

The imaging device 400 may receive one or more of the prepared tissue block and/or the prepared tissue slice and capture corresponding image data. In some embodiments, the imaging device 400 may capture image data corresponding to a plurality of prepared tissue slices and/or a plurality of prepared tissue blocks. The imaging device 400 may further capture, through the lens of the imaging apparatus 402, using the image sensor of the imaging apparatus 402, a representation of a prepared tissue slice and/or a prepared tissue block as placed on the platform. Therefore, the imaging device 400 can capture image data in order for the image analysis system to compare the image data to determine if the image data corresponds to the same tissue block.

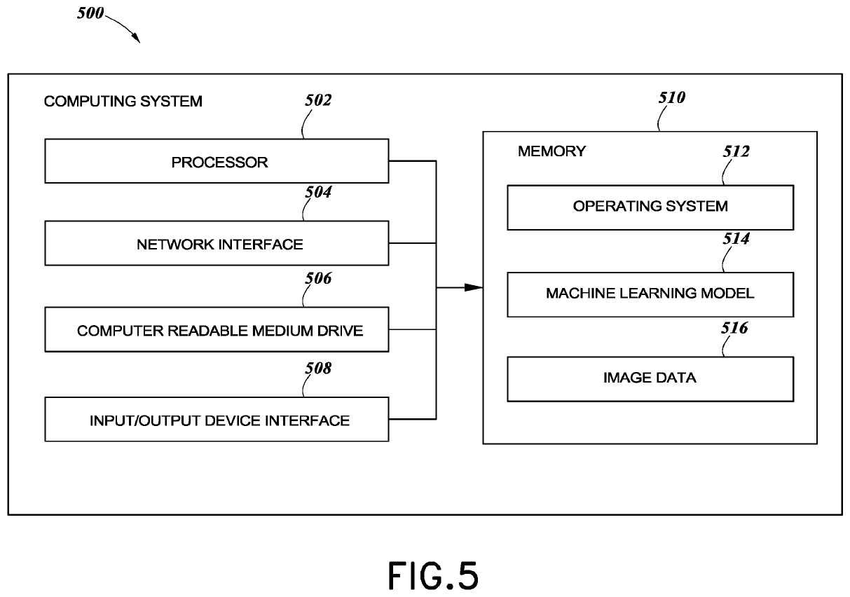

FIG. 5 is an example computing system 500 which can implement any one or more of the imaging device 102, image analysis system 108, and user computing device 110 of the imaging system illustrated in FIG. 1. The computing system 500 may include: one or more computer processors 502, such as physical central processing units (“CPUs”); one or more network interfaces 504, such as a network interface cards (“NICs”); one or more computer readable medium drives 506, such as a high density disk (“HDDs”), solid state drives (“SDDs”), flash drives, and/or other persistent non-transitory computer-readable media; an input/output device interface 508, such as an input/output (“TO”) interface in communication with one or more microphones; and one or more computer readable memories 510, such as random access memory (“RAM”) and/or other volatile non-transitory computer-readable media.

The network interface 504 can provide connectivity to one or more networks or computing systems. The computer processor 502 can receive information and instructions from other computing systems or services via the network interface 504. The network interface 504 can also store data directly to the computer-readable memory 510. The computer processor 502 can communicate to and from the computer-readable memory 510, execute instructions and process data in the computer readable memory 510, etc.

The computer readable memory 510 may include computer program instructions that the computer processor 502 executes in order to implement one or more embodiments. The computer readable memory 510 can store an operating system 512 that provides computer program instructions for use by the computer processor 502 in the general administration and operation of the computing system 500. The computer readable memory 510 can further include computer program instructions and other information for implementing aspects of the present disclosure. For example, in one embodiment, the computer readable memory 510 may include a machine learning model 514 (also referred to as a machine learning algorithm). As another example, the computer-readable memory 510 may include image data 516. In some embodiments, multiple computing systems 500 may communicate with each other via respective network interfaces 504, and can implement multiple sessions each session with a corresponding connection parameter (e.g., each computing system 500 may execute one or more separate instances of the method 700), in parallel (e.g., each computing system 500 may execute a portion of a single instance of the method 700), etc.

### Machine Learning Algorithms

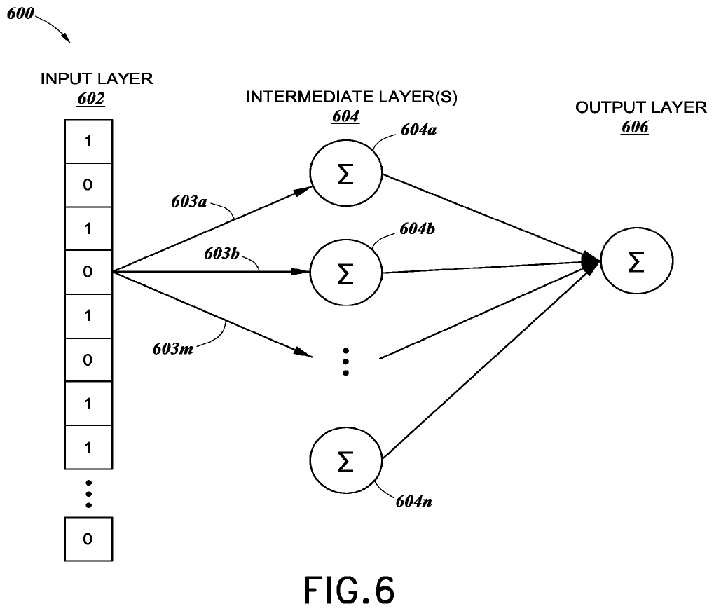

FIG. 6 depicts a schematic diagram of a machine learning algorithm 600, including a multiple layer neural network in accordance with aspects of the present disclosure. The machine learning algorithm 600 can include one or more machine learning algorithms in order to diagnose one or more diseases within image data provided as an input to the machine leaning algorithm 600 by identifying structures or features present in the image data that are consistent with training data used to train the machine learning algorithm 600. Further, the machine learning algorithm 600 may correspond to one or more of a machine learning model, a convolutional neural network, etc.

The machine learning algorithm 600 can include an input layer 602, one or more intermediate layer(s) 604 (also referred to as hidden layer(s)), and an output layer 606. The input layer 602 may be an array of pixel values. For example, the input layer may include a 320×320×3 array of pixel values. Each value of the input layer 602 may correspond to a particular pixel value. Further, the input layer 602 may obtain the pixel values corresponding to the image. Each input of the input layer 602 may be transformed according to one or more calculations.

Further, the values of the input layer 602 may be provided to an intermediate layer 604 of the machine learning algorithm. In some embodiments, the machine learning algorithm 600 may include one or more intermediate layers 604. The intermediate layer 604 can include a plurality of activation nodes that each perform a corresponding function. Further, each of the intermediate layer(s) 604 can perform one or more additional operations on the values of the input layer 602 or the output of a previous one of the intermediate layer(s) 604. For example, the input layer 602 is scaled by one or more weights 603a, 603b, . . . , 603m prior to being provided to a first one of the one or more intermediate layers 604. Each of the intermediate layers 604 includes a plurality of activation nodes 604a, 604b, . . . , 604n. While many of the activation nodes 604a, 604b, . . . are configured to receive input from the input layer 602 or a prior intermediate layer, the intermediate layer 604 may also include one or more activation nodes 604n that do not receive input. Such activation nodes 604n may be generally referred to as bias activation nodes. When an intermediate layer 604 includes one or more bias activation nodes 604n, the number m of weights applied to the inputs of the intermediate layer 604 may not be equal to the number of activation nodes n of the intermediate layer 604. Alternatively, when an intermediate layer 604 does not includes any bias activation nodes 604n, the number m of weights applied to the inputs of the intermediate layer 604 may be equal to the number of activation nodes n of the intermediate layer 604.

By performing the one or more operations, a particular intermediate layer 604 may be configured to produce a particular output. For example, a particular intermediate layer 604 may be configured to identify an edge of a tissue sample and/or a block sample. Further, a particular intermediate layer 604 may be configured to identify an edge of a tissue sample and/or a block sample and another intermediate layer 604 may be configured to identify another feature of the tissue sample and/or a block sample. Therefore, the use of multiple intermediate layers can enable the identification of multiple features of the tissue sample and/or the block sample. By identifying the multiple features, the machine learning algorithm can provide a more accurate identification of a particular image. Further, the combination of the multiple intermediate layers can enable the machine learning algorithm to better diagnose the presence of a disease. The output of the last intermediate layer 604 may be received as input at the output layer 606 after being scaled by weights 605a, 605b, 605m. Although only one output node is illustrated as part of the output layer 606, in other implementations, the output layer 606 may include a plurality of output nodes.

The outputs of the one or more intermediate layers 604 may be provided to an output layer 606 in order to identify (e.g., predict) whether the image data is indicative of a disease present in the tissue sample. In some embodiments, the machine learning algorithm may include a convolution layer and one or more non-linear layers. The convolution layer may be located prior to the non-linear layer(s).

In order to diagnose the tissue sample associated with image data, the machine learning algorithm 600 may be trained to identify a disease. By such training, the trained machine learning algorithm 600 is trained to recognize differences in images and/or similarities in images. Advantageously, the trained machine learning algorithm 600 is able to produce an indication of a likelihood that particular sets of image data are indicative of a disease present in the tissue sample.

Training data associated with tissue sample(s) may be provided to or otherwise accessed by the machine learning algorithm 600 for training. The training data may include image data corresponding to a tissue sample tissue block data that has previously been identified as having a disease. The machine learning algorithm 600 trains using the training data set. The machine learning algorithm 600 may be trained to identify a level of similarity between first image data and the training data. The machine learning algorithm 600 may generate an output that includes a representation (e.g., an alphabetical, numerical, alphanumerical, or symbolical representation) of whether a disease present in a tissue sample corresponding to the first image data.

In some embodiments, training the machine learning algorithm 600 may include training a machine learning model, such as a neural network, to determine relationships between different image data. The resulting trained machine learning model may include a set of weights or other parameters, and different subsets of the weights may correspond to different input vectors. For example, the weights may be encoded representations of the pixels of the images. Further, the image analysis system can provide the trained image analysis module 600 for image processing. In some embodiments, the process may be repeated where a different image analysis module 600 is generated and trained for a different data domain, a different user, etc. For example, a separate image analysis module 600 may be trained for each data domain of a plurality of data domains within which the image analysis system is configured to operate.

Illustratively, the image analysis system may include and implement one or more imaging algorithms. For example, the one or more imaging algorithms may include one or more of an image differencing algorithm, a spatial analysis algorithm, a pattern recognition algorithm, a shape comparison algorithm, a color distribution algorithm, a blob detection algorithm, a template matching algorithm, a SURF feature extraction algorithm, an edge detection algorithm, a keypoint matching algorithm, a histogram comparison algorithm, or a semantic texton forest algorithm. The image differencing algorithm can identify one or more differences between first image data and second image data. The image differencing algorithm can identify differences between the first image data and the second image data by identifying differences between each pixel of each image. The spatial analysis algorithm can identify one or more topological or spatial differences between the first image data and the second image data. The spatial analysis algorithm can identify the topological or spatial differences by identifying differences in the spatial features associated with the first image data and the second image data. The pattern recognition algorithm can identify differences in patterns of the first image data and the training data. The pattern recognition algorithm can identify differences in patterns of the first image data and patterns of the training data. The shape comparison algorithm can analyze one or more shapes of the first image data and one or more shapes of the second image data and determine if the shapes match. The shape comparison algorithm can further identify differences in the shapes.

The color distribution algorithm may identify differences in the distribution of colors over the first image data and the second image data. The blob detection algorithm may identify regions in the first image data that differ in image properties (e.g., brightness, color) from a corresponding region in the training data. The template matching algorithm may identify the parts of first image data that match a template (e.g., training data). The SURF feature extraction algorithm may extract features from the first image data and the training data and compare the features. The features may be extracted based at least in part on particular significance of the features. The edge detection algorithm may identify the boundaries of objects within the first image data and the training data. The boundaries of the objects within the first image data may be compared with the boundaries of the objects within the training data. The keypoint matching algorithm may extract particular keypoints from the first image data and the training data and compare the keypoints to identify differences. The histogram comparison algorithm may identify differences in a color histogram associated with the first image data and a color histogram associated with the training data. The semantic texton forests algorithm may compare semantic representations of the first image data and the training data in order to identify differences. It will be understood that the image analysis system may implement more, less, or different imaging algorithms. Further, the image analysis system may implement any imaging algorithm in order to identify differences between the first image data and the training data.

### Diagnosis of a Disease Using a Combination of Virtual Staining and Chemical Staining of a Tissue Sample

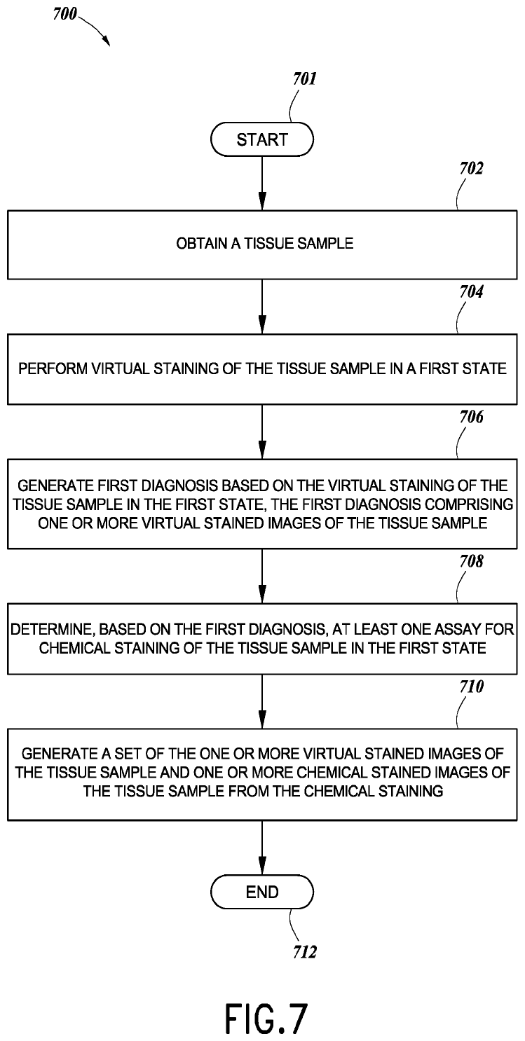

FIG. 7 is an example method 700 for hybrid virtual and chemical staining of tissue samples in accordance with aspects of this disclosure. As described above, virtual staining can be used to diagnose a disease based on a tissue sample. However, there may be certain limitations to virtual staining; for example, virtual staining may not be able to generate certain markers used to differentiate between different types of a given disease. Thus, after reviewing images of the virtually stained tissue sample, a pathologist may order one or more chemical stains which can help distinguish between the possible types of the disease indicated by the virtually stained images.

Traditional virtual imaging techniques may use coverslipped slides of the tissue sample, which may limit the automated downstream staining of the coverslipped slides. Accordingly, the pathologist will order downstream chemically stained samples manually, adding downtime to the histology process. In addition, the chemical staining is performed on a separate section of the tissue sample, limiting the ability of the pathologist to view the same cells with multiple markers, and requiring a larger piece of tissue that can be sliced to prepare multiple slides and requiring additional labor.

Aspects of this disclosure, and the method 700 FIG. 7 in particular, can address at least some of the above drawbacks of the traditional virtual staining techniques. One or more of the blocks 702-710 of the method 700 may be performed by an imaging system, such as the image analysis system 104 of FIG. 1. However, depending on the implementation, one or more of the blocks 702-710 may be implemented as a computing system (e.g., the computing system 500 of FIG. 5), etc.

With reference to FIG. 7, the method 700 starts at block 701. At block 702, the method 700 involves obtaining a tissue sample. At block 704, the method 700 involves performing virtual staining of the tissue sample in a first state to generate one or more virtual stained images of the tissue sample. The first state may include the tissue sample in an unadulterated state (e.g., the tissue sample has not been permanently coverslipped or chemically stained). In some implementations the first state may include the use of a non-permanent coverslipping method that can be removed, while in other implementations the tissue sample is not coverslipped in the first state.

At block 706, the method 700 involves generating a first diagnosis based on the virtual staining of the tissue sample in the first state. The first diagnosis includes the one or more virtual stained images of the tissue sample. The first diagnosis may include an initial primary diagnosis such as the identification of a tumor within the tissue sample.

In certain implementations, the first diagnosis may be obtained using a machine learning algorithm (e.g., the machine learning algorithm 600 of FIG. 6). For example, the method 700 can involve executing a machine learning algorithm using the virtual stained images of the tissue sample as an input. The machine learning algorithm can be configured to generate the first diagnosis including an indication of the disease based on the tissue sample.

At block 708, the method 700 involves determining, based on the first diagnosis, at least one assay for chemical staining of the tissue sample in the first state. In some embodiments, the hardware processor may automatically identify, order, or cause one or more chemical stains of the tissue sample based on the first diagnosis (e.g., automating the chemical staining of the tissue sample and/or automatically ordering, obtaining, or accessing the chemical staining of the tissue sample). As used herein, the term automatically generally refers to a process performed without any user input. In response to the order for chemical stain(s), automated robotics or a lab technician may transfer and chemically stain the tissue sample. In some implementations, the transfer and chemical staining of the tissue sample involves performing parallel or sequential multiplexing (e.g. dissolvable chromogen) on the tissue sample.

In some implementations, when multiple different chemical stains (e.g., using different assays) of the tissue sample are ordered it may be possible to strip at least one of the ordered chemical stains from the tissue sample without significantly damaging the tissue sample. In these implementations, the method 700 may further involve stripping a first one of the chemical stains from the tissue sample and staining the tissue sample with a second one of the ordered chemical stains. Thus, the method 700 may involve performing a plurality of chemical stains on the tissue sample, and where possible, stripping and re-staining the same tissue sample. When a multiple chemical stains cannot be stripped from the tissue sample without damaging the tissue sample, the method 700 may involve slicing the tissue sample to prepare multiple slides, each of which can be stained with a different chemical stain, which can be done without the pathologist's review and the associated delay.

In some implementations, the machine learning algorithm may follow a decision tree that selects one or more assays for chemical staining based on the disease indicated by the first diagnosis. The chemical staining of the tissue sample using the assay(s) is configured to aid a pathologist differentiating between different types of the disease indicated by the first diagnosis.

As described above, the first diagnosis (e.g., an initial primary diagnosis) can be obtained by providing image data generated by the virtual staining to a machine learning algorithm. Based on the identification of the first diagnosis, the machine learning algorithm can further follow a decision tree that facilitates selecting the at least one assay based on the disease indicated by the first diagnosis. One example of a simplified decision tree is a follows: if the primary diagnosis shows A, run stains B, C, D; otherwise run stains E, F. In some implementations, the method 700 may involve automatically ordering the chemical staining of the tissue sample in response to the machine learning algorithm generating the first diagnosis.

The machine learning may also be able to make more accurate diagnoses of tissue sample under certain circumstances. For example, a machine learning algorithm can access a relatively large pool of knowledge generated based on a relatively large set of images/diagnoses from pathologists to improve diagnoses. That is, the machine learning algorithm may be able to process an amount of data that is not practical for the pathologist to review, and thus, may be able to make inferences that would not be practical for a pathologist.

In some implementations, the machine learning algorithm may use other inputs in addition to the virtual stained images in generating the first diagnosis. Example additional source(s) of data which can be used as input(s) include: patient history, clinical notes, and/or other testing data.

In other implementations, at block 708 a system (e.g., the imaging system 104, the computing system 500, or component(s) thereof) and/or pathologist may review the first diagnosis including the one or more virtual stained images to determine the at least one assay for chemical staining of the tissue sample. In one example, the pathologist may not wish to rely on the machine learning algorithm or the machine learning algorithm may not have sufficient training data to generate the first diagnosis. Thus, the pathologist may manually review the virtually stained images and order one or more chemical stains of the tissue sample which may be useful in diagnosing a disease in the tissue sample.

At block 710, the method 700 involves generating a set of the one or more virtual stained images of the tissue sample generated from the virtual staining and the one or more chemical stained images of the tissue sample generated from the chemical staining. For example, the set of virtual image(s) and chemical stained image(s) can be collated along with the first diagnosis into a complete package which is provided to the pathologist to diagnose the tissue sample. The pathologist may be able to overlay one or more markers of interest on the virtual stained and chemical stained images. The method 700 ends at block 712.

There are a number of advantages to aspects of this disclosure over the traditional histology workflow. According to aspects of this disclosure, the same piece of tissue can be used for every stain (virtual and chemical), which allows for easy overlay of markers on the images produced. That is, when the same piece of tissue is used for all of the stains, the same cellular structure may be present in each of the images, allowing the pathologist to view the same cellular structure as stained using various different techniques (e.g., virtual staining and chemical staining using one or more assays).

Additionally, aspects of this disclosure can significantly reduce the amount of delays in waiting for the lab to complete a test. From the perspective of the pathologist, there may be little to no delays associated with waiting for the lab to perform test(s) since the pathologist may have all or most of the required information to complete a diagnosis in the complete package. Because the pathologist received the complete package including all of the typically ordered images based on the first diagnosis, the pathologist's workload is reduced to compared to reviewing an initial slide (e.g., an H&E stained slide) and ordering subsequent slides used for determining the type of the diagnosis.

Further aspects of this disclosure may involve the use of coverslipless imaging (e.g., imaging that does not involve using coverslipped slides of the tissue sample) to generate the virtual stained image(s) and/or the chemical stained images, which can reduce or eliminate manual handling of the tissue sample, thereby significantly reducing the chance of damaging the tissue sample.

The use of hybrid virtual and chemical staining can also improve the laboratory workflow by eliminating the need to cut additional sections of the tissue sample without an order against them thereby reducing the risk of lost productivity. In addition, the lab does not need to store and retrieve tissue sample blocks and slides as often using aspects of the hybrid approach described herein compared to traditional histology workflow. The use of hybrid virtual and chemical staining can further reduce the risk of running out of tissue samples since same section of the tissue sample can provide a large amount of data. This can be particularly advantageous when the tissue sample obtained from a patient is relatively small, for example, the size of some smaller biopsies may limit the number of slides that can be prepared (e.g., sample may have a thickness allowing for 2-3 slides to be prepared).

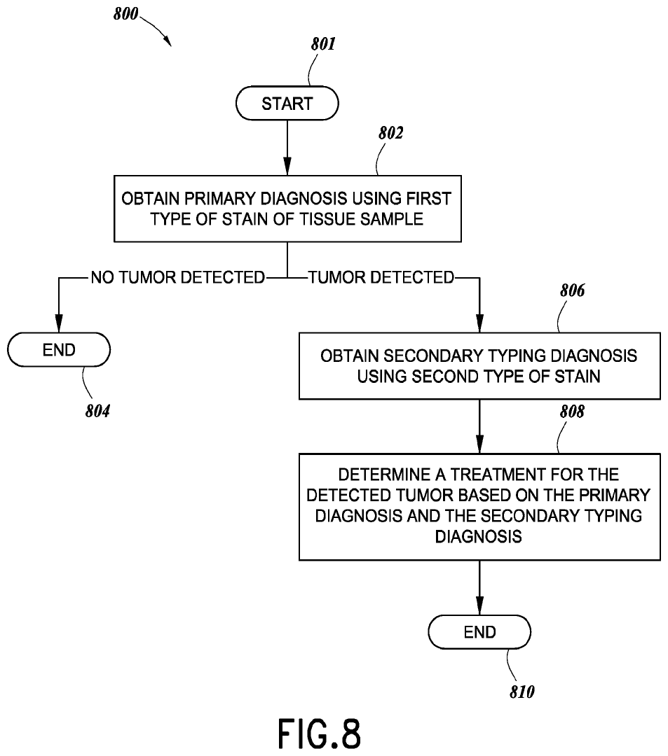

FIG. 8 is an example method 800 for diagnosing and typing a disease in accordance with aspects of this disclosure. In detail, the typing of a primary diagnosis may involve obtaining a primary diagnosis using a first type of stain and typing the primary diagnosis using one or more secondary stains. This structure may form a decision tree used to order one or more chemical stains for typing the primary diagnosis. In aspects of this disclosure, the example decision tree may be implemented by the machine learning algorithm, for example, at block 708 of FIG. 7.

One or more of the blocks 802-808 of the method 800 may be performed by an imaging system, such as the image analysis system 104 of FIG. 1. However, depending on the implementation, one or more of the blocks 802-808 may be implemented by a lab technician, a pathologist, a computing system (e.g., the computing system 500 of FIG. 5), etc.

With reference to FIG. 8, the method 800 starts at block 801. At block 802, the method 800 involves obtaining a primary diagnosis using a first type of stain of the tissue sample. If no tumor is detected, the method 800 ends at block 804. If a tumor is detected, at block 806 the method 800 involves obtaining a secondary typing diagnosis using a second type of stain.

In one example, the primary diagnosis may be an identification of breast cancer based on the use of an H&E staining of a tissue sample. One way in which breast cancer can be typed is by determining HER2 status using an IHC stain. The results of the IHC stain may indicate whether a particular treatment (e.g., Herceptin treatment) may be effective, or whether a subsequent test (e.g., FISH stain) should be ordered to determine whether the particular treatment may be effective.

At block 808, the method 800 involves determining a treatment for the detected tumor based on the primary diagnosis and the secondary typing diagnosis. Block 808 may be performed at least partially by an oncologist based on the primary diagnosis and the secondary typing diagnosis. The method 800 ends at block 810. It is noted that the example described with reference to FIG. 8 is for illustrative purposes only, and that the method of 800 may relate to detecting other types of conditions or cancers, and different examples of typing applicable for the given condition or cancer.

### CONCLUSION

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the disclosure should not be taken to imply that the terminology is being re-defined herein to be restricted to including any specific characteristics of the features or aspects of the technology with which that terminology is associated.

It will be appreciated by those skilled in the art that various modifications and changes can be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures can be combined, interchanged or excluded from other embodiments.

With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

Directional terms used herein (e.g., top, bottom, side, up, down, inward, outward, etc.) are generally used with reference to the orientation shown in the figures and are not intended to be limiting. For example, the top surface described above can refer to a bottom surface or a side surface. Thus, features described on the top surface may be included on a bottom surface, a side surface, or any other surface.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “comprising” as used herein is synonymous with “including,” “containing,” or “characterized by,” and is inclusive or open-ended and does not exclude additional, unrecited elements or method steps.

The above description discloses several methods and materials of the present invention(s). This invention(s) is susceptible to modifications in the methods and materials, as well as alterations in the fabrication methods and equipment. Such modifications will become apparent to those skilled in the art from a consideration of this disclosure or practice of the invention(s) disclosed herein. Consequently, it is not intended that this invention(s) be limited to the specific embodiments disclosed herein, but that it cover all modifications and alternatives coming within the true scope and spirit of the invention(s) as embodied in the attached claims.

1. An image analysis apparatus, comprising:
a memory coupled to an imaging device; and
a hardware processor coupled to the memory and configured to:
receive image data from the imaging device, the image data representative of a tissue sample in a first state,
perform virtual staining of the tissue sample based on the image data to generate one or more virtual stained images of the tissue sample,
execute an artificial intelligence algorithm using the one or more virtual stained images of the tissue sample as an input, the artificial intelligence algorithm configured to generate a first diagnosis comprising an indication of a disease based on the tissue sample,
automatically identify one or more types of chemical stains based on the indication of the disease generated by the artificial intelligence algorithm, and
automatically order chemical staining of the tissue sample in the first state based on the identified one or more types of chemical stains.

2. The apparatus of claim 1, wherein the hardware processor is further configured to:
receive one or more chemically stained images, and
generate a set of the one or more virtual stained images of the tissue sample from the virtual staining and the one or more chemically stained images of the tissue sample from the chemical staining.

3. The apparatus of claim 1, wherein the hardware processor is further configured to:
generate a first diagnosis based on the virtual staining of the tissue sample in the first state, the first diagnosis comprising the one or more virtual stained images of the tissue sample.

4. The apparatus of claim 1, wherein the hardware processor is further configured to:
determine at least one assay for the chemical staining of the tissue sample in the first state,
wherein the order of the chemical staining includes an indication of the at least one assay to be used in the chemical staining of the tissue sample.

5. The apparatus of claim 2, wherein the hardware processor is further configured to:
generate a first diagnosis based on the virtual staining of the tissue sample in the first state,
wherein the chemical staining of the tissue sample using the at least one assay is configured to differentiate between different types of the disease indicated by the first diagnosis.

6. The apparatus of claim 1, wherein:
the machine learning algorithm is configured to follow a decision tree that selects the at least one assay based on the disease indicated by the first diagnosis.

7. The apparatus of claim 2, wherein the chemical staining is performed on the same tissue sample used in the virtual staining.

8. The apparatus of claim 2, wherein the hardware processor is further configured to perform the virtual staining and the generating of the set of the one or more images without storing the tissue sample.

9. The apparatus of claim 2, wherein:
the imaging device is configured to generate the image data using coverslipless imaging, and
the chemical staining is imaged using coverslipless imaging.

10. The apparatus of claim 1, wherein automatically identifying the one or more types of chemical stains and automatically ordering the chemical staining of the tissue sample are performed without receiving user input.

11. A method of diagnosing a disease based on a tissue sample, comprising:
performing virtual staining of the tissue sample in a first state to generate one or more virtual stained images of the tissue sample;
executing an artificial intelligence algorithm using the virtual stained images of the tissue sample as an input;
automatically generating a first diagnosis comprising an indication of a disease based on an output of the artificial intelligence algorithm, the first diagnosis comprising the one or more virtual stained images of the tissue sample;
automatically determining, based on the first diagnosis, at least one assay for chemical staining of the tissue sample in the first state; and
generating a set of the one or more virtual stained images of the tissue sample from the virtual staining and one or more chemical stained images of the tissue sample from the chemical staining.

12. The method of claim 11, wherein the chemical staining is performed on the same tissue sample used in the virtual staining.

13. The method of claim 11, wherein the virtual staining and the generating the set of the one or more images are performed without storing the tissue sample.

14. The method of claim 11, further comprising:
generating image data of the tissue sample using an image device,
wherein the image data generated by the image device is used as an input for the virtual staining.

15. The method of claim 14, wherein the generating of the image data and the chemical staining are performed using coverslipless imaging.

16. An image analysis apparatus, comprising:
a memory coupled to an imaging device; and
a hardware processor coupled to the memory and configured to:
obtain image data from the imaging device, the image data representative of a tissue sample,
perform virtual staining of the tissue sample based on the image data to generate one or more virtual stained images of the tissue sample,
execute an artificial intelligence algorithm using the one or more virtual stained images of the tissue sample as an input, the artificial intelligence algorithm configured to generate a first diagnosis comprising an indication of a disease based on the tissue sample, and
obtain, based on indication of the disease, one or more images of the same tissue sample having a chemical stain.

17. The apparatus of claim 16, wherein the tissue sample is directed to undergo the chemical stain after the hardware processor performs virtual staining of the tissue sample.

18. The apparatus of claim 16, wherein the hardware processor is further configured to cause the ordering of the chemical staining of the tissue based on the one or more virtual stained images of the tissue sample.

19. The apparatus of claim 16, wherein the hardware processor is further configured to generate a first diagnosis based on the virtual staining of the tissue sample.

20. (canceled)
